(19) United States
(12) Patent Application Publication
(10) Pub. No.: US 2026/0100289 A1
(43) Pub. Date: Apr. 9, 2026

(54) DENTAL COLLABORATION CLOUD

(71) Applicant: Friction Free Dental, Inc., Boston, MA (US)

(72) Inventor: Kevin Farrell, Boston, MA (US)

(21) Appl. No.: 19/348,014

(22) Filed: Oct. 2, 2025

Related U.S. Application Data
(60) Provisional application No. 63/704,821, filed on Oct. 8, 2024.

(51) Int. Cl. G16H 80/00; G16H 30/20; G16H 30/40

(52) U.S. Cl. CPC G16H 80/00; G16H 30/20; G16H 30/40

(57) ABSTRACT

A system and method for providing dental collaboration. A system is disclosed that includes: a virtual practice private network (VPPN) service configured to establish a unique VPPN for each of a plurality of dental practices, wherein each VPPN grants access rights to a unique set of users; an adaptable integration framework (AIF) configured to provide read and write capabilities to disparate practice management systems utilized by the plurality of dental practices; a universal clinical toolset service configured to allow client applications to interact with data from the disparate practice management systems via a universal dental interface (UDI), wherein the UDI includes a system agnostic charting interface, a system agnostic notes interface, a system agnostic periodontics interface, and a system agnostic imaging interface; and a collaboration service configured to allow the unique set of users of an associated VPPN to capture and share information in a collaboration interface within the UDI.

### PRIORITY CLAIM

This application claims priority to copending provisional application DENTAL COLLABORATION CLOUD, filed Oct. 8, 2024, Ser. No. 63/704,821, the contents of which are hereby incorporated by reference.

### TECHNICAL FIELD

The subject matter of this disclosure relates to a computing infrastructure that supports secure information processing, collaboration and presentation within the dental field.

### BACKGROUND

Practice management systems (PMSs) in the dental field are utilized to streamline operations of the practice and are often capable of managing everything from patient records, charting and treatment plans to scheduling, billing and claims processing. While such systems are powerful tools that can handle a wide range of tasks, there are inherent limitations in such systems. For example, those engaged in the dental field often need to share information and collaborate with others outside of the practice to implement a treatment plan. For example, a dentist may need to discuss an issue with a specialist regarding a particular patient. Because there exist any number of different PMS's and other legacy systems, they are generally not designed to securely share information outside of the practice. Accordingly, collaboration is typically done in an ad hoc manner, e.g., via telephone calls or email, which limits the collaboration process.

### SUMMARY

Aspects of the disclosure provide a computing infrastructure for enhancing practice management systems and other legacy systems used in the dental field (collectively “PMSs”) to allow for better collaboration and patient care.

A first aspect provides a dental collaboration system, comprising: a virtual practice private network (VPPN) service configured to establish a unique VPPN for each of a plurality of dental practices, wherein each VPPN grants access rights to a unique set of users; an adaptable integration framework (AIF) configured to provide read and write capabilities to disparate practice management systems utilized by the plurality of dental practices; a universal clinical toolset service configured to allow client applications to interact with data from the disparate practice management systems via a universal dental interface (UDI), wherein the UDI includes a system agnostic charting interface, a system agnostic notes interface, a system agnostic periodontics interface, and a system agnostic imaging interface; and a collaboration service configured to allow the unique set of users of an associated VPPN to capture and share information in a collaboration interface within the UDI.

A second aspect provides a cloud computing infrastructure, comprising: a memory; and a processor coupled to the memory and configured to provide dental collaboration according to a process that includes: configuring a unique VPPN for each of a plurality of dental practices, wherein each VPPN grants access rights to a unique set of users; providing an adaptable integration framework (AIF) configured to provide read and write capabilities to disparate practice management systems utilized by the plurality of dental practices; communicating dental practice data with client applications via a universal dental interface (UDI), wherein the dental practice data include patient records obtained from the disparate practice management systems, and wherein the UDI includes a system agnostic charting interface, a system agnostic notes interface, a system agnostic periodontics interface, and a system agnostic imaging interface; and sharing information among the unique set of users of an associated VPPN via a collaboration interface within the UDI.

A third aspect provides a method of providing dental collaboration, comprising: configuring a unique VPPN for each of a plurality of dental practices, wherein each VPPN grants access rights to a unique set of users; providing an adaptable integration framework (AIF) configured to provide read and write capabilities to disparate practice management systems utilized by the plurality of dental practices; communicating dental practice data with client applications via a universal dental interface (UDI), wherein the dental practice data include patient records obtained from the disparate practice management systems, and wherein the UDI includes a system agnostic charting interface, a system agnostic notes interface, a system agnostic periodontics interface, and a system agnostic imaging interface; and sharing information among the unique set of users of an associated VPPN via a collaboration interface within the UDI.

Other aspects include any one of those noted herein, and wherein the access rights granted to the unique set of users for an associated VPPN determines which of the disparate practice management systems are accessible to the unique set of users.

Still other aspects include any of those noted herein, and wherein the system agnostic charting interface overlays patient specific image data onto a generic graphical image of a set of teeth based on patient records obtained via the AIF.

Further aspects include any of those noted herein, and wherein the AIF utilizes one of application programming interfaces (APIs) or robotic process automation (RPA) to read and write data to disparate practice management systems.

Additional aspects include any of those noted herein, and wherein the collaboration interface provides an active capture facility for recording and sharing screen displays, annotations and voice recordings among the unique set of users for the associated VPPN, and wherein the active capture facility can record and share screen displays presented in the UDI.

More aspects include any of those noted herein, and wherein the collaboration interface includes a messaging facility for sharing messages with other users in the unique set of users within the associated VPPN.

Other aspects include any of those noted herein, and wherein at least one user of the unique set of users for the associated VPPN is an external specialist or lab that can only access patient records from an associated practice management system via the UDI.

Still other aspects include any of those noted herein, and further comprising a data normalizer that converts patient records from the disparate practice management systems to a common format utilized by the UDI, and wherein data normalizer includes a dental code normalizer that maps vendor code extensions and practice code extensions to a normalized format.

### BRIEF DESCRIPTION OF THE DRAWINGS

These and other features of this invention will be more readily understood from the following detailed description of the various aspects of the invention taken in conjunction with the accompanying drawings in which:

FIG. 1 shows a collaborative computing infrastructure having a dental collaboration cloud (DCC) according to embodiments.

FIG. 2 depicts virtual practice private network services according to embodiments.

FIG. 3 depicts an illustrative set of virtual practice private networks according to embodiments.

FIG. 4 depicts a data normalizer according to embodiments.

FIG. 5 depicts an adaptable integration framework according to embodiments.

FIG. 6 shows a DCC client according to embodiments.

FIG. 7 shows a charting interface according to embodiments.

FIG. 8 shows a further view of a charting interface according to embodiments.

FIG. 9 depicts an imaging interface according to embodiments.

FIG. 10 depicts a periodontics interface according to embodiments.

FIG. 11 depicts a notes interface according to embodiments.

FIG. 12 shows a collaboration overview operating in a private network according to embodiments.

FIG. 13 depicts a collaboration interface according to embodiments.

FIG. 14 depicts an active capture process according to embodiments.

FIG. 15 depicts a further view of a collaboration interface according to embodiments.

FIG. 16 shows a network according to embodiments.

FIG. 17 shows a computing system according to embodiments.

The drawings are not necessarily to scale. The drawings are merely schematic representations, not intended to portray specific parameters of the invention. The drawings are intended to depict only typical embodiments of the invention, and therefore should not be considered as limiting the scope of the invention. In the drawings, like numbering represents like elements.

### DETAILED DESCRIPTION

Various embodiments provided herein relate to a computing system, cloud infrastructure, and method for enhancing practice management systems and other legacy systems used in the dental field (collectively “PMSs”) to allow for better collaboration and patient care. In one aspect a solution is provided that allows uniform viewing and sharing of information amongst users that utilize disparate PMSs within dental related practices. In certain aspects, information is presented in an interactive and graphical platform that displays data in a format independent of the underlying PMSs from which data is collected. Additionally, interactions with graphical and audio information (e.g., voice and video inputs, annotations, screen navigation, etc.) by a first user can be captured (i.e., recorded) and forwarded to a second user for playback in a secure manner. Interactions by the second user can likewise be captured and sent back to the first user in a secure manner, thus providing a system agnostic asynchronous collaboration platform. These technical enhancements provide a highly effective and efficient platform to create/capture and playback such collaborations asynchronously, by enabling users to easily speak and interact with content as they would in-person thus allowing for a more efficient productive use of participants'time who no longer need to coordinate synchronous collaborations together. Related aspects are described in additional detail in U.S. patent application Ser. No. 19/172,846, entitled ASYNCHRONOUS DATA SHARING COMPUTING INFRASTRUCTURE, filed on Apr. 8, 2025, the contents of which are hereby incorporated by reference.

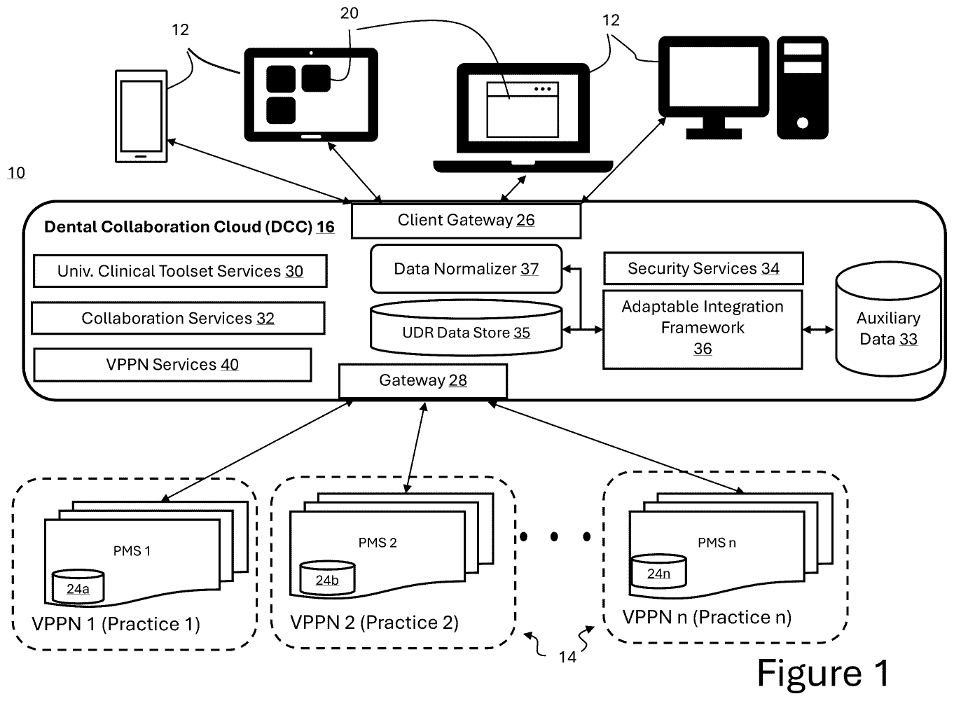

Referring to FIG. 1, an illustrative dental collaborative computing infrastructure 10 is depicted configured to enhance existing PMSs (e.g., PMS 1, PMS 2, etc.) within associated dental practices 14 (e.g., Practice 1, Practice 2, etc.). Infrastructure 10 is managed by a dental collaboration cloud (DCC) 16, which may for example be implemented as a software as a service (SaaS) network, cloud, server, and/or other system(s). DCC 16 includes a number of services that allow users operating client devices 12 to more effectively interact with patient data and collaborate with others, both within and external to the particular practice.

As shown, each dental practice 14 may include one or more PMSs that generally comprise closed databases 24a, b, . . . n (or generally “database 24”), which are only accessible by an associated practice 14. DCC 16 creates a virtual provider private network (VPPN) around each practice that provides a secure platform which allows users to seamlessly share data and collaborate with other users either within or external to their particular practice. For example, a dentist may need to share data from their PMS and collaborate with an endodontist that does not have access to the dentist's PMS. DCC 16 provides a set of services that allows participants to securely view and share private data in a system agnostic manner via client devices 12. Rather than directly interfacing with individual PMSs, client devices 12 interact with DCC 16, which provides a secure gateway to PMS data of existing practices 14. Each PMS is connected to the DCC 16 via a backend gateway 28, e.g., using an API (application programming interface) or RPA (robotic process automation), which allows PMS data from each practice to be read from or written to DCC 16 in an as needed manner.

DCC 16 accordingly allows users to collaborate and share system data from respective practices 14 via client devices 12 that connect to DCC 16 via a client gateway 26 (e.g., an API or the like). Client devices 12 may comprise any type of computing device, e.g., a smartphone, a tablet, a laptop, a desktop, etc. Collaboration and sharing on a client device 12 may occur via a DCC client 20 that may include a downloadable application, a browser, or any other interactive technology.

As noted, DCC 16 includes a number of services that allow for enhancement, collaboration and sharing. Some of the core services include: universal clinical toolset (UCT) services 30 that provide common tools utilized within a practice; collaboration services 32 that provide a mechanism for capturing and sharing mixed media information; security services 34 that provide a platform for controlling system and data access to information; an adaptable integration framework 36 that provides a data processing platform for processing, reading and writing data from disparate PMSs; a data normalizer 37 for normalizing data to a common format and allowing for a consistent and universal user clinical toolset experience, across the mix of PMS's; and VPPN services 40 for implementing private networks. DCC 16 also includes an auxiliary data store 33 that stores auxiliary patient information beyond what is stored by the practice's PMS, e.g., recordings, transcriptions, collaborations, etc.

As noted, access to PMS data by DCC 16 is achieved via a backend gateway 28, which may in certain cases use an API. However, in cases of older legacy systems without any API or interface to access data, the DCC 16 can utilize techniques such as an RPA (Robotic Process Automation)-like approach to directly interact with one or more legacy system User Interfaces (UI), to perform operations directly by automatically navigating through the application, to extract, save or update data from/to the legacy system.

In order to provide a secure infrastructure for sharing information, DCC 16 includes a set of virtual practice private network (VPPN) services 40 that establishes a unique VPPN for each practice 14 within which information can be selectively shared.

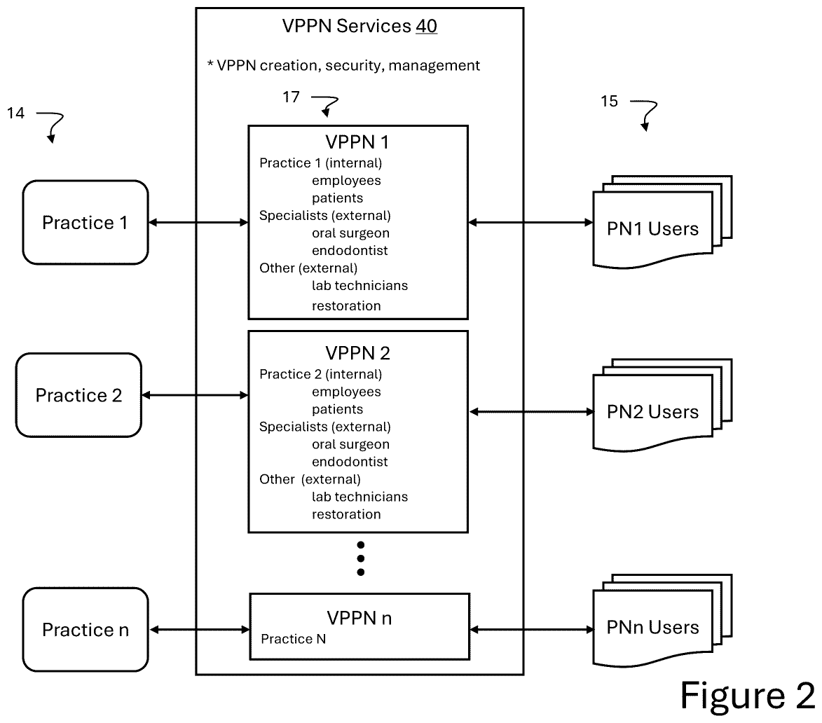

VPPN creation can be done using any known technology to, e.g., generate and install security certificates and keys, configure the cloud to route traffic, set up ports, configure virtual network clients, etc. FIG. 2 depicts an illustrative embodiment of VPPN services 40 configured to provide private network creation, security, management, etc., for respective practices 14. As shown, each practice 14 has an associated private network 17 (VPPN 1, VPPN 2, etc.), whose users 15 for example include: individuals internal to the practice, e.g., employees and patients, etc.; specialists external to the practice, e.g., oral surgeons, endodontists, etc. ; and other external entities, e.g., lab technicians, restoration companies, etc. The selection of users 15 in each private network 17 is determined by the respective practice 14, which can set access controls (e.g., privileges and security levels) for each user. For example, a dentist in the practice may have full access to all records, whereas a billing clerk may have limited access. Similarly, external users may only have messaging and collaboration privileges, and not access to the practice's PMS data.

Accordingly, each private network 17 provides an associated practice 14 a secure platform to both allow and control access to data both internal and external users to the practice.

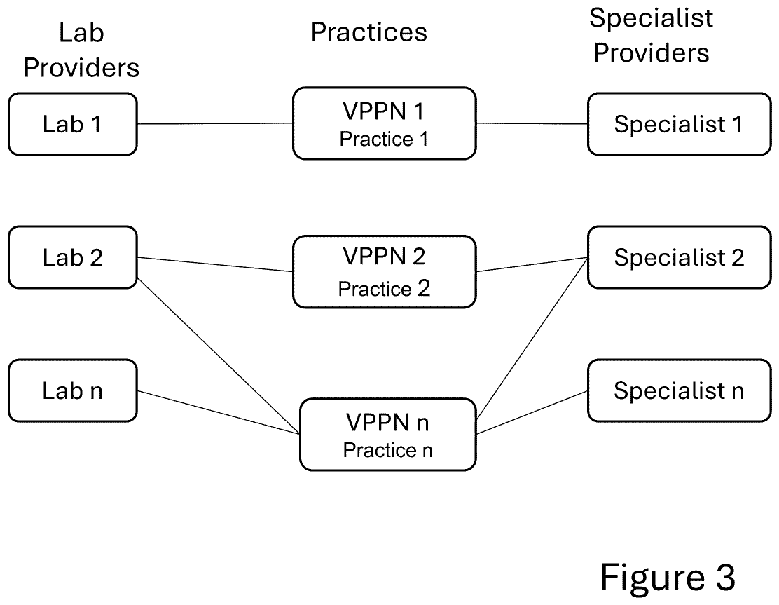

FIG. 3 depicts an example of how VPPNs allow different practices to incorporate external providers into their private network. Each dental practice has a set of providers both internal to the practice (e.g., dentists, hygienist assistants, front desk) and external to the practice (e.g., dental laboratories, specialists such as endodontists, a periodontist, an oral surgeon, an orthodontist, etc.). A VPPN is created for each practice allowing user-controlled access to their practice management system (PMS) data (e.g., patient data) and external provider data (e.g., specialists and/or labs). Access to the data is controlled by the VPPN for each practice to ensure that data is secure and only available to users with the necessary credentials. As can be seen, a specialist or a lab may be members of more than one VPPN, e.g., special 2 is part of VPPN 2 and VPPN n.

In order to provide a consistent experience, data normalizer 37 normalizes data (e.g., patient records, file formats, images, etc.) into a common UDR format that is stored in a UDR data store 35 (FIG. 1). For example, data normalizer 37 may convert text, pdf, or spreadsheet-based patient records obtained from disparate PMSs into a normalized or common format, using, e.g., comma-separated values (.csv), tab-separated values (.tsv) or XML. This common format can then be used to display information by the universal clinical toolset 52 described herein.

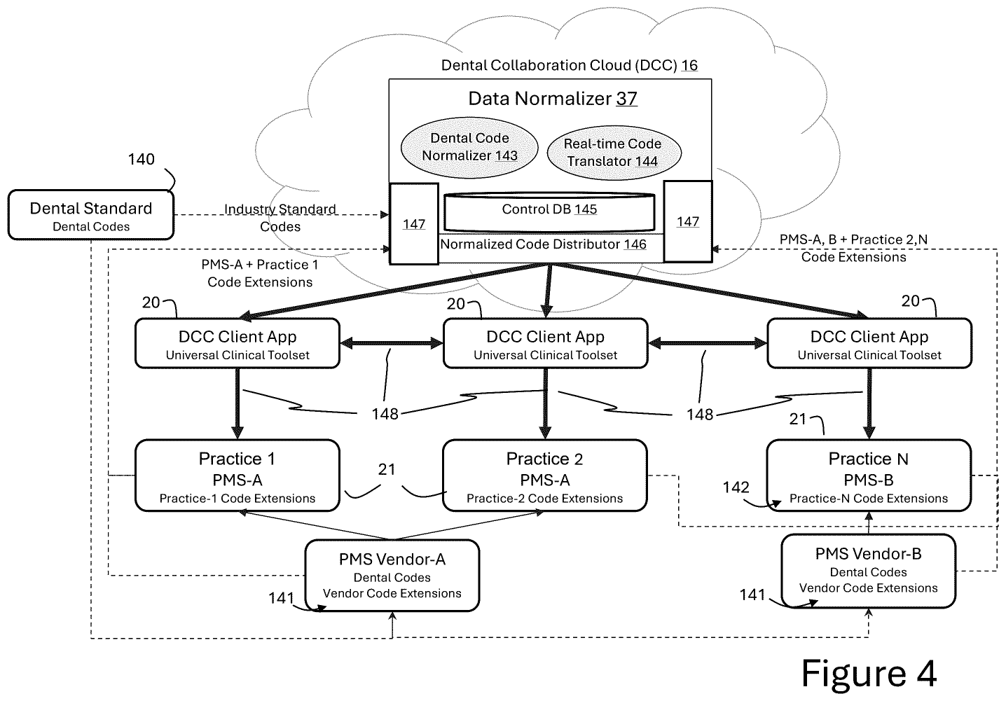

FIG. 4 depicts an illustrative overview of certain functionality of data normalizer 37. As shown, data normalizer 37 includes computing modules for normalizing dental code data across the mix of PMS and practice code differences and custom extensions. Within the field of dentistry, there are standard dental codes 140. In addition, PMS vendors have code extensions 141 specific to their PMS platform, and individual practices may further use their own practice code extensions 142. Data normalizer 37 includes a dental code collector 147 for collecting industry standard codes 140, vendor code extensions 141, and practice code extensions 142. Data normalizer 37 further includes a dental code normalizer 143 and real-time code translator 144 that translate and normalize code extensions into a normalized or common format, regardless of the PMS platform or practice codes being used. These codes are stored in a control database (DB) 145 and distributed back the various practices via DCC client apps 20 using normalized code distributor 146. Control DB 145 maintains all the dental codes and extensions, mappings between normalized and vendor/practice extensions, dependencies, etc. Accordingly, communications 148 between client apps 20 and PMS's 21 can utilize common normalized extensions.

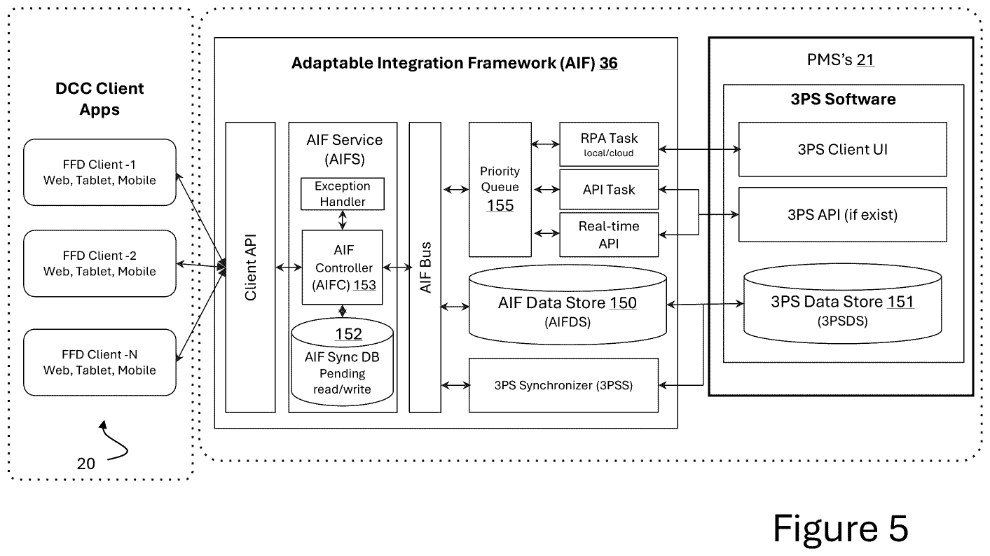

FIG. 5 depicts an illustrative implementation of an adaptable integration framework (AIF) 36, which provides client apps 20 with a high-performance real-time user experience and data consistency with disparate PMSs 21 running 3rd Party System (3PS) software. AIF 36 implements a system for reading and writing dental practice data to and from disparate PMS's 21, handling 3PS software integration/application programming interface (API) capabilities or lack thereof. In the case where the PMS includes a 3PS API, AIF uses an API task module or real-time API module to exchange data between the PMS and a priority queue 155. In the case where PMS 21 does not include an API, data is exchanged using an RPA task module. Other exchange mechanisms could likewise be implemented as needed.

In some embodiments, AIF 36 builds and maintains an AIF Data Store (AIFDS) 150, to mirror the contents of one or more 3PS Data Stores (3PSDS) 151 in the PMS 21. Thus, the AIF 36 can be configured where the AIFDS 150 or the 3PSDS 151 operate as the Database of Record (DoR) with the 3PS synchronizer ensuring data consistency. The AIF 36 also ensures client apps 20 are always working with the most recent data/updates, including pending updates, per the AIF Sync DB (i.e., cache) 152.

AIF 36 performs the following:
Creates and maintains a working copy of any legacy 3rd party system data in the AIF Data Store 150.
Maintains a 3rd Party system (PMS, Imaging, etc.) as database of record, ensuring eventual consistency.
Provides client apps 20 high performance response for reads and writes.
Utilizes an AIF Controller 153 to assess all changes/updates, priority, pre-check-in sync, and maintains eventual consistency between an AIF cache (i.e., DB 152) and 3PS system data.
Manages all data exchanges and synchronizations with each PMS 21, monitors the AIF Cache and PMS, and notifies AIFC 153 of changes.
The AIF and PMS may be on the same or different host computers (connected by high speed network).

For example, Client-1 updates patient-A data and client-N submits a request to view Patient-A data. The AIF 36 would return the latest view of Patient-A, reflecting the update from client-1, even if that client-1 update to the PMS 21 is pending in the Sync DB 152.

AIF 36 allows the DCC client apps 20 to be the primary clinical toolset used and source of all information. AIF 36 provides eventual consistency, but gives priority to the priority queue 155. AIF services integrate with the data normalizer (FIG. 5) to ensure data is converted to and from a common format. To store, manage and provide a primary datastore for DCC client tools, AIF 36 effectively operates as a clinical PMS.
Charting: DCC add treatments, update treatment status, etc.
Imaging—FFD image gallery will provide access to all images, across all sources of images in the practice.
Perio—create and view all exams.
Clinical notes—create and view all clinical notes.

### Collaboration—Unique to DCC.

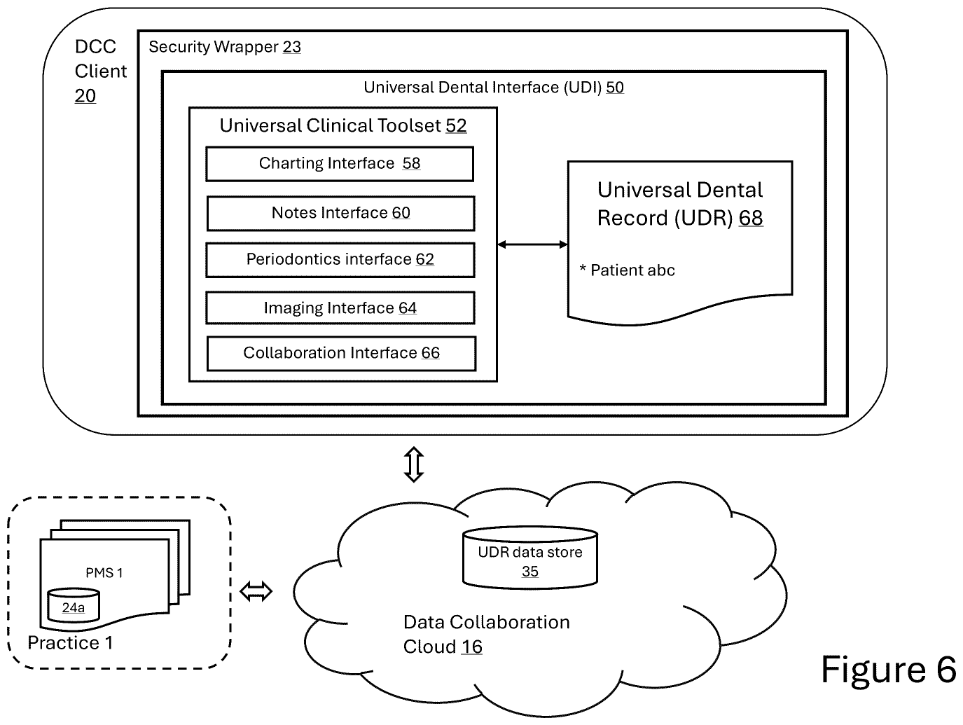

FIG. 6 depicts an illustrative embodiment of a DCC client 20 configured to allow user interaction with patient data obtained from a practice via DCC 16. Each DCC client 20 includes a security wrapper 23 that ensures that the user has privileges to access respective patient information (e.g., based on private network rights, privilege levels, etc.). The security wrapper 23 is for example implemented and controlled by local security features and/or security services 34 provided by DCC 16.

The DCC client 20 displays patient information within a universal dental interface (UDI) 50, which is an agnostic platform for interactively displaying a universal dental record (UDR) 68 and other information for a selected patient. The universal dental record 68 is created by the adaptable integration framework (AIF) 36 within DCC 16 based on information from a respective PMS and is temporally saved in UDR data store 35.

Accordingly, regardless of the PMS or other system used by the practice, the information will always be transformed and displayed in a standardized manner within UDI 50.

Within UDI 50 is universal clinical toolset 52, which not only provides standard PMS interfaces such as a charting interface 58, a notes interface 60, a periodontics interface 62, and an imaging interface 64, but also provides a collaboration interface 66. Accordingly, when a user such as dentist within Practice 1 wishes to see a patent record via the DCC client 20, the dentist signs in and requests a patient record (e.g., via a search feature not shown). The request is passed to DCC 16, which in turn securely obtains the record from the dentist's PMS (e.g., PMS 1). DCC 16 then transforms the record into a standardized UDR format and stores the record in a UDR data store 35. The patient's UDR 68 is then uploaded to the UDI 50 on client 20, where the dentist can view, edit, and interact with the record 68. Any changes or updates to record 68 are returned back to DCC 16, which converts the changes back to the native format of PMS 1 and writes the changes thereto. Note that the update process can occur in real time, near real time, in batch mode, or at a later time. Accordingly, users can view and interact with patient records in a standardized matter, regardless of the client device or underlying PMS utilized by the practice.

It is understood that DCC client 20 and/or UDI 50 may be implemented in any manner. For example, they may be implemented as virtual applications optimized to run in a virtual environment that can reside on the client device and/or in the cloud. In other cases, they may be implemented as web applications that run within a browser. Still in other cases, they may be implemented as native programs running on a device.

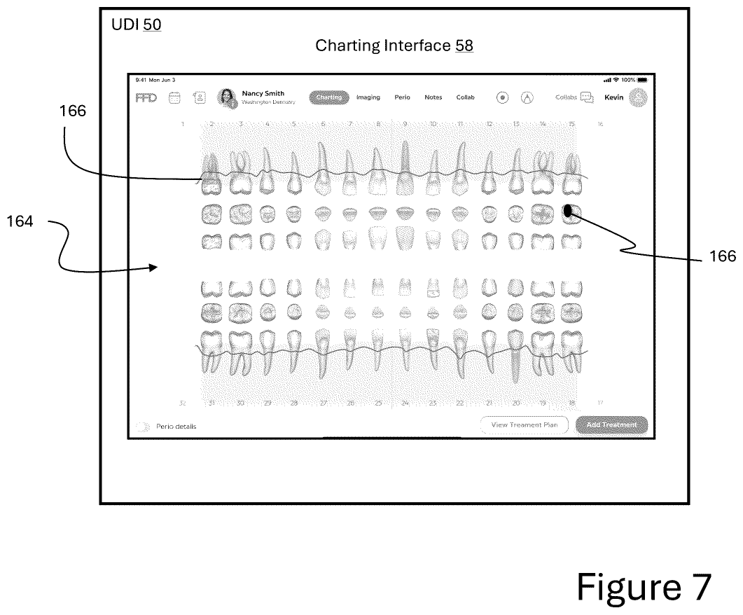

FIG. 7 depicts an illustrative charting interface 58 within UDI 50. In this case, interface 58 includes a generic graphical image 164 (i.e., a chart of a set of teeth) and patient specific image data 166 overlayed (i.e., treatment plan in red, existing completed work in green, etc.) on the generic graphical image 164. Patient specific image data 166 is for example obtained via a patient record access service that is implemented by AIF 36 (FIGS. 1 and 5), which makes a call to backend gateway 28 to retrieve records from one or more PMSs. The overlay procedure processes the retrieved data and creates and renders the patient specific image data 166 (e.g., gum lines, cavities, tooth number, treatment type, treatment type attributes) with, for instance, different colors indicating planned/red versus existing/green, etc.) on the graphical image 164.

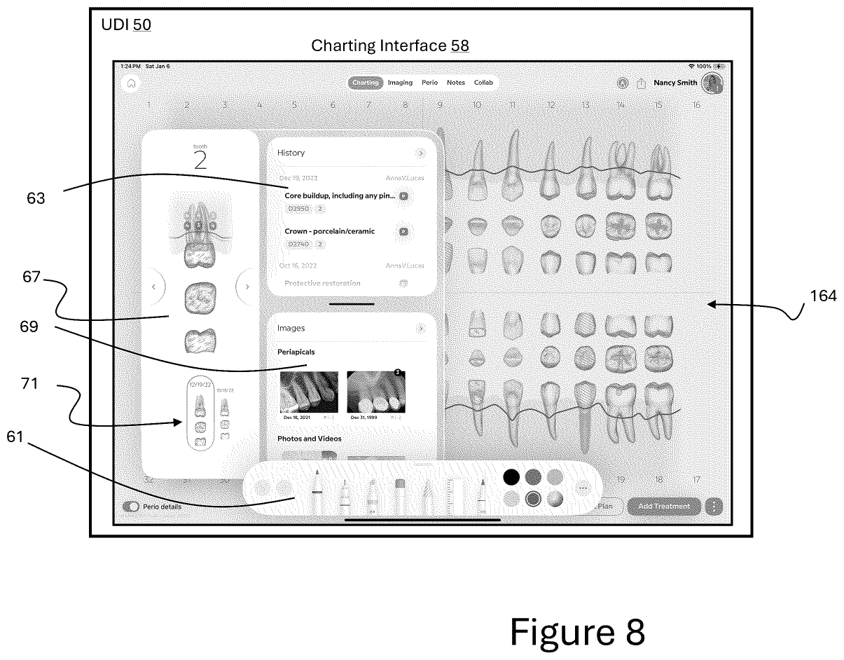

As shown in FIG. 8, the user is able to select and interact with various elements of the image 164 within the charting interface 58. For example, the user is able to select a tooth (e.g., with a touch screen or mouse action) and navigate to more detailed images and data. For example, more granular details of a selected tooth are presented in new dialog windows 67, 69. When such a selection occurs, AIF 36 and backend gateway 28 (FIG. 1) make several different calls to the respective PMS(s), aggregating all the information shown in a single consolidated window 67. In addition, within window 67, a history selector 71 allows for the full display to repopulate according to a selected date or appointment to generate and show what the selected tooth state looked like at any given appointment. For example, a dentist may be interested when a tooth was extracted and likewise, what the condition of the tooth was leading up to and need for the tooth to be extracted. This history is being automatically generated by DCC 16 from a visit ledger and history. In addition, a mark-up tool 61 is provided for marking up and annotating screen images. Also shown is a charting notes window 63 for viewing and inputting notations for the selected tooth.

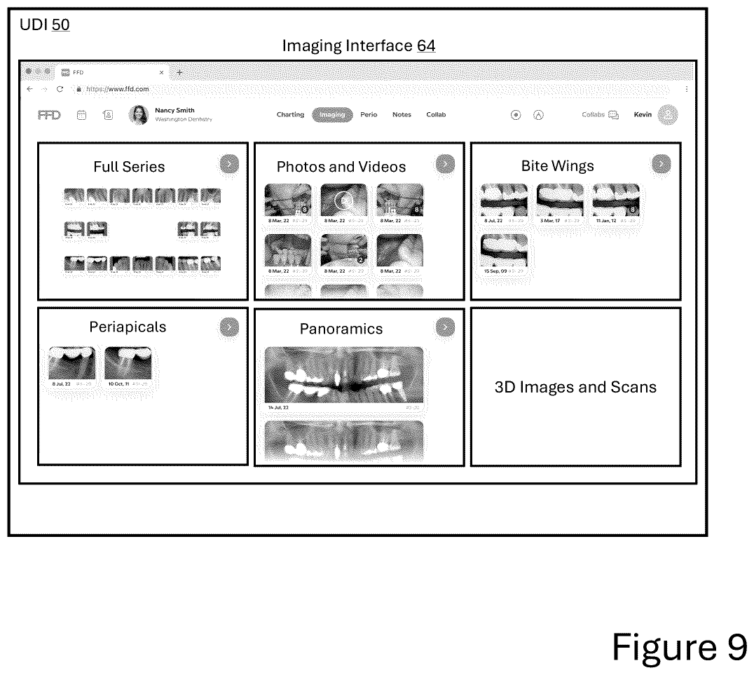

FIG. 9 depicts an illustration of an imaging interface 64, which allows the user to view and interact with different images files from one or more system databases 24, e.g., JPEGs, video files, audio files, 3D files (i.e., MRI), etc. Often, such image files reside in a legacy system separate from a practice's primary PMS (e.g., in an MRI database, various different independent imaging equipment databases, photos stored on a memory card on a SLR camara, a photo library on smartphone, etc.). Imaging interface 64 accordingly allows such imaging data to be presented and shown in a single aggregate view in the UDI 50, independent of the source or location of the disparate independent databases where the images are stored. AIF 36 and backend gateway 28 (FIG. 1) manage the collection of the various images from their respective disparate sources and aggregate the view for presentation in interface 64. In the example shown, imaging interface 64 depicts full series, photos and videos, bite wings, periapicals, panoramics, and 3D images and scans.

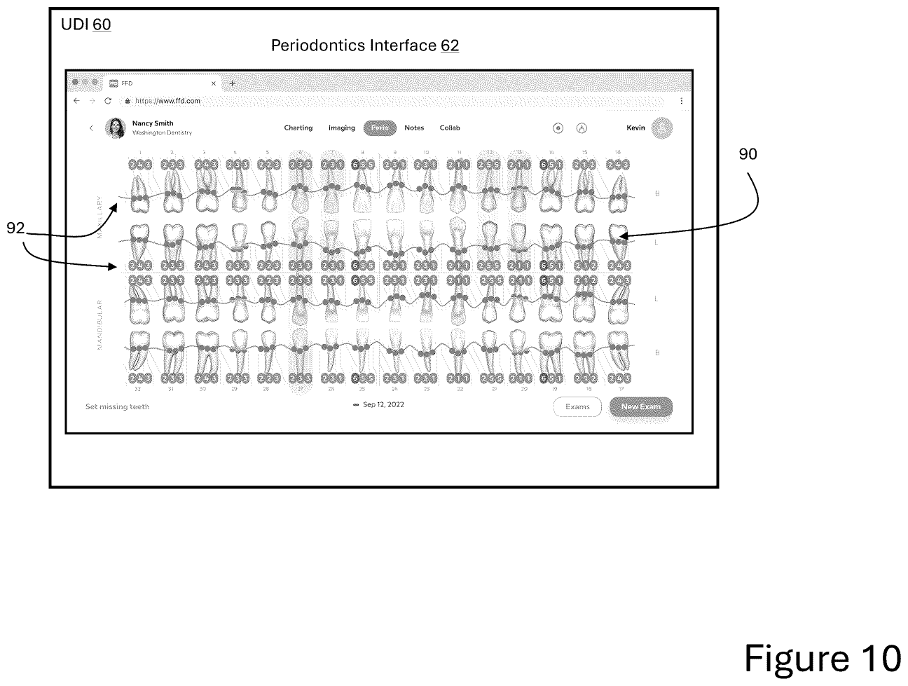

FIG. 10 depicts an illustrative periodontics interface 62, which renders periodontics data 92 (i.e., gum health) for a selected patient record overlayed on top of an interactive graphic tooth image 90. Periodontics interface 62 operates similarly to the charting interface 58, but depicts a different set of graphical information within the dentistry domain. In one illustrative embodiment, a voice interface tool is provided that allows a user to make data entries (e.g., gum measurements) using voice input that will appear in the diagram and be saved to a system database.

Various components of the universal clinical toolset 52 can integrate and leverage other related services, such as transcription services, audio storage, notes processing services, etc. For example, captured natural language (NL) inputs can be stored in a persistent storage to allow a user to later hear the original input. Additionally, NL inputs can be transcribed into text.

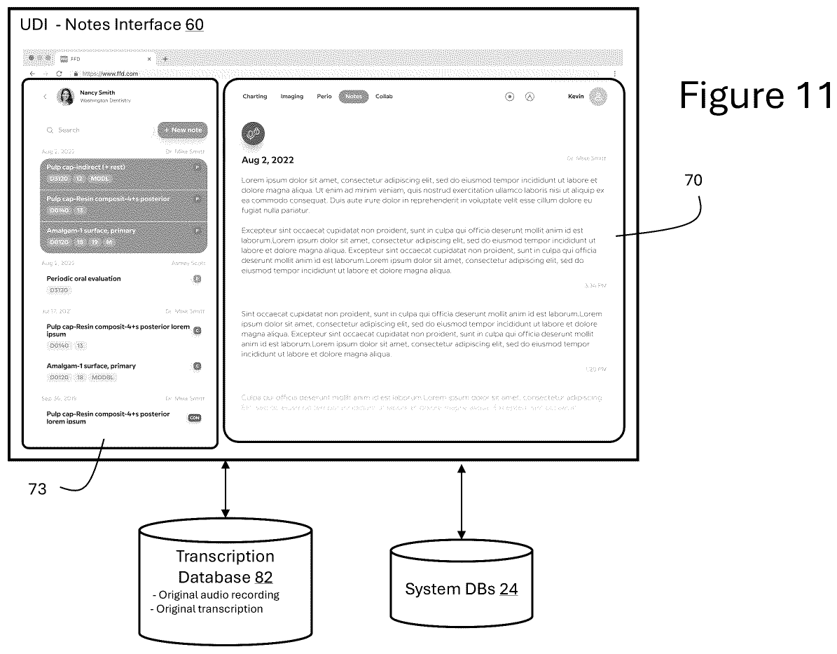

FIG. 11 depicts views of a notes interface 60, which allows user transcriptions to be captured and displayed. In this example, a note 70 is depicted in the right window and a notes summary 73 for the patient is depicted in the left window. From the notes summary 73, the user can search through existing notes, select a note for display, or create a new note. When creating a new note, a dentist can dictate patient examination details, which are transcribed and stored in transcription database 82. In other aspects, an entire exam can be recorded start to finish in a note and stored in a system database 24.

Notes may include any information that the user wants to view or create regarding a presented data record (e.g., in the context of dental, patient feedback, patient findings, patient treatment plans, patient work completed that day, etc.) Thus, from the notes interface 60, the user can review existing notes from one or more system databases 24.

Accordingly, notes from one or more PMS systems can be viewed, edited, created and saved. Notes, such as medical notes regarding a patient, can be generated either by typing or via a voice transcription system that generates text based on voice input. In various aspects, transcription database 82 (e.g., stored as auxiliary data 33 in DCC 16) may be utilized to store the voice/audio input, e.g., as a redundancy to the generated text.

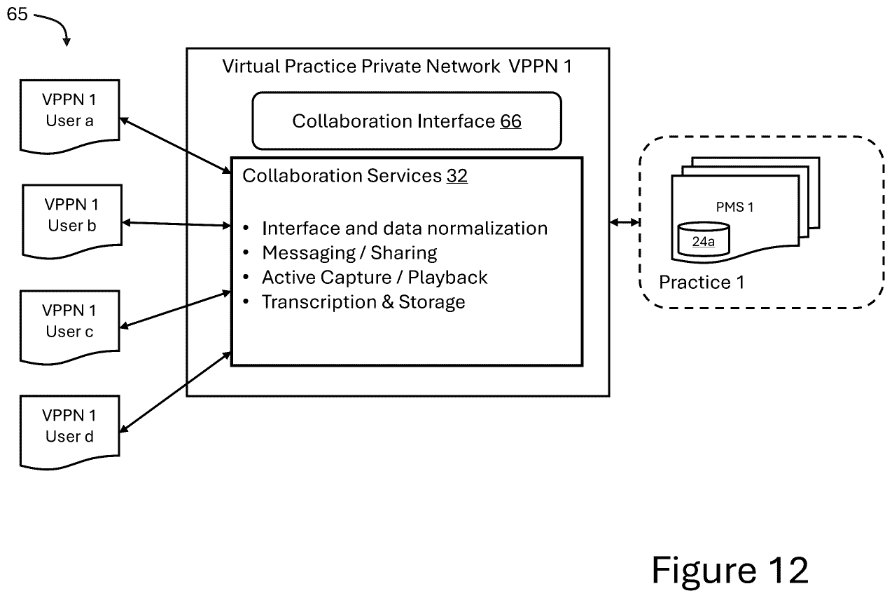

As noted, in addition to standard tools described above, universal clinical toolset 52 (FIG. 6) includes a collaboration interface 66, which operates in conjunction with collaboration services 32 in DCC 16 (FIG. 1). FIG. 12 depicts an overview of a collaboration implementation within a private network VPPN 1. In this example, VPPN 1 is established for a dental practice (Practice 1) to allow for collaboration among a set of predefined users 65 in a secure environment (both internal and external to Practice 1).

Collaboration interface 66 provides a platform in which system data and user analysis (e.g., navigating through UDI screens, annotations, etc.) can be integrated and shared with other users within VPPN 1. Within collaboration interface 66, users can also actively capture interactions performed within (or outside) universal dental interface 50, including screen recording of any application on the device that is displayed to a user, again with annotations, voice inputs, video inputs, etc. The recording of such information, referred to herein as an “active capture,” can then be shared with other users via collaboration interface 66 and collaboration services 32 provided by DCC 16. In addition, the collaboration interface 66 also provides transcription and storage services, whereby users can playback in silence for privacy and all active captures are automatically transcribed and can be viewed with sub-titles displaying the transcription.

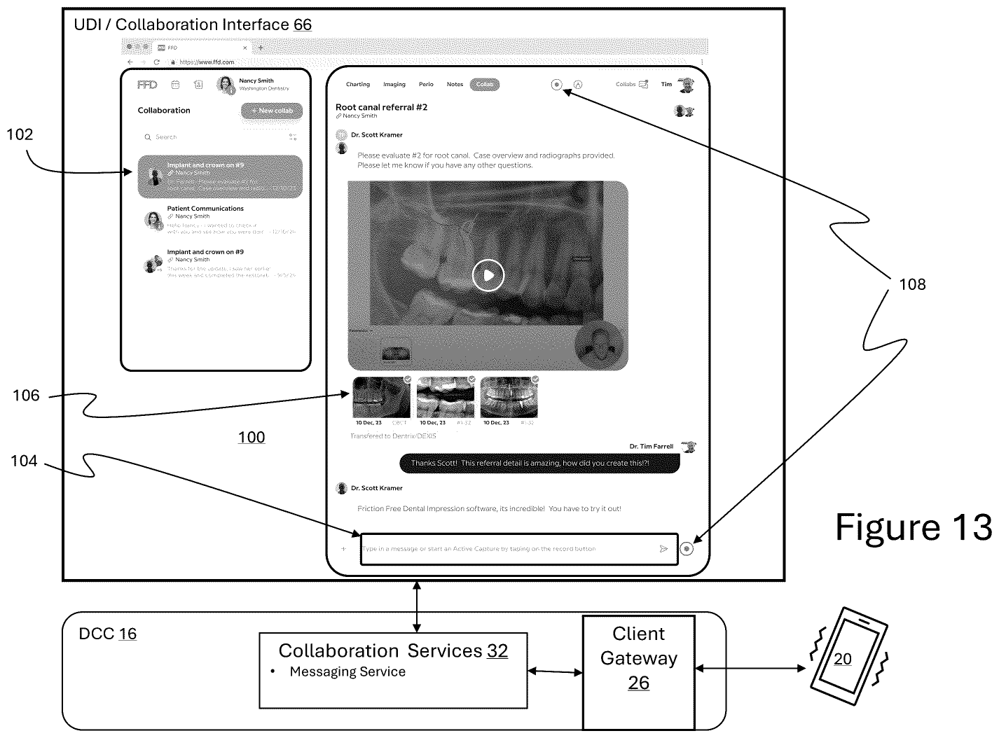

FIG. 13 depicts an illustrative collaboration interface 66 that includes a messaging platform 100. One the lefthand side of platform 100 is a list of users 102 within the current VPPN that have been previously messaged for collaboration. New users can be searched and selected as well. Once selected, the user can view existing messages, as well as create/send a collaboration message, e.g., enter text 104 and attach files 106 (e.g., image files). Additionally, the user can also launch an “active capture” with button 108, described in further detail herein. Once the message is composed, it can be sent to a selected user for review via a messaging service within collaboration services 32 via client gateway 26. A notification will be sent and the receiving user will receive a notification within their client 20.

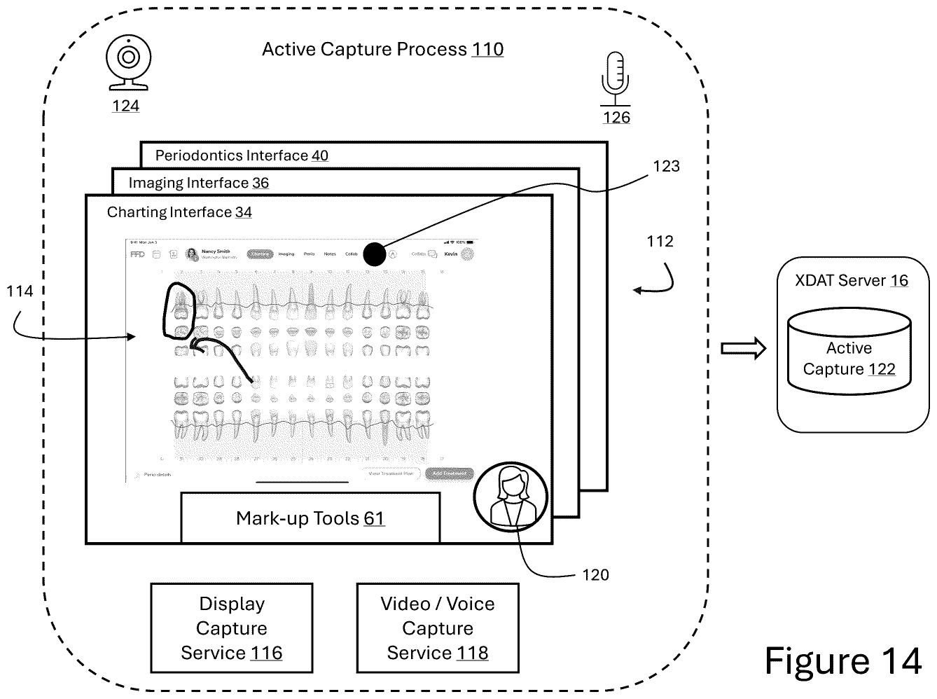

FIG. 14 depicts an example of an active capture process 110. The active capture process 110 allows a user to capture their actions while interacting within the DCC client/device (as well as interactions outside a DCC client). In this example, the process 110 includes: (1) a display capture service 116 that will capture and record what is being viewed on the user's device screen (e.g., charting interface 114); and (2) a video/voice capture service 118 that will capture and record both video and audio of the user. The resulting information is packaged, transcribed (e.g., in subtitles) and output as an active capture 122, that can be stored or shared.

In this example, the user launches an active capture process 110 (e.g., by clicking a record button 123). As the user navigates through various screens and windows 112, display capture service 116 records the interaction as a streaming video. Additionally, a set of mark-up tools 61 are provided that allows the user to markup what is being displayed, which is also captured by display capture service 116. At the same time, video of the user is captured by camera 124 and displayed in window 120, and audio of the user is captured by microphone 126 (by video/voice capture service 118). The resulting captured information is packaged into an audio/video (AV) file that is outputted as active capture 122 to the DCC 16. Accordingly, when the active capture 122 is played back, it will show the screens 112 as the user viewed them, annotations the user made, as well as the user's audio and video inputs.

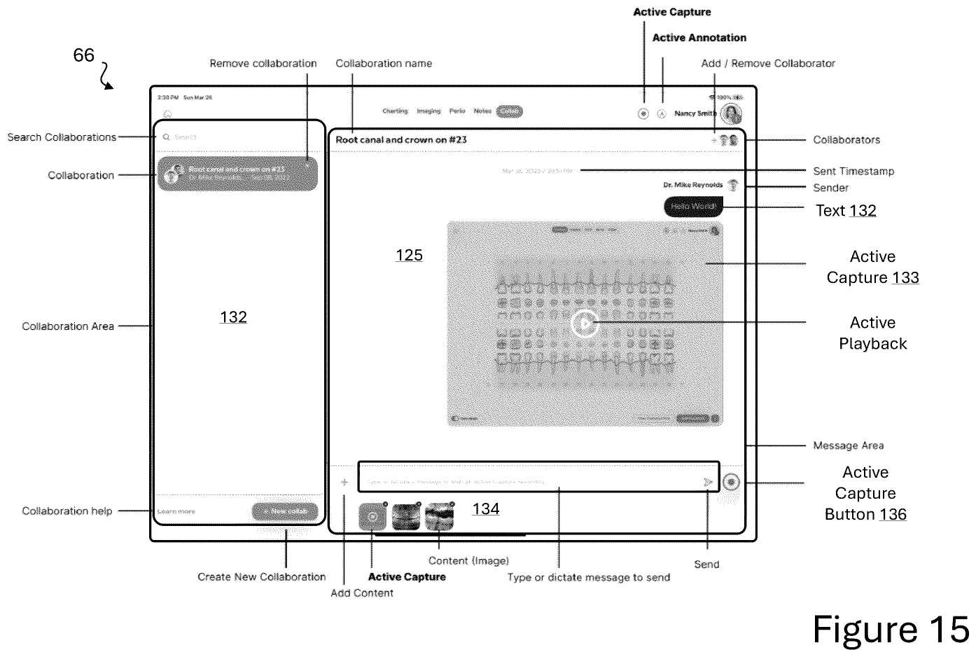

FIG. 15 depicts the collaboration interface 66 at a receiving user's client. From within interface 66, the user can view received/sent messages in a messaging window 125, including text messages 132, an active capture 133, and other images, as well as thumbnails 134. From with messaging window 125, the user can also view an active playback of the active capture 122. The receiving user can also initiate a responsive active capture by clicking an active capture button 136, allowing the receiving user to record their own active capture, e.g., containing audio, video, screen displays, annotations, etc. The resulting responsive active capture can then be sent back to the original sending user.

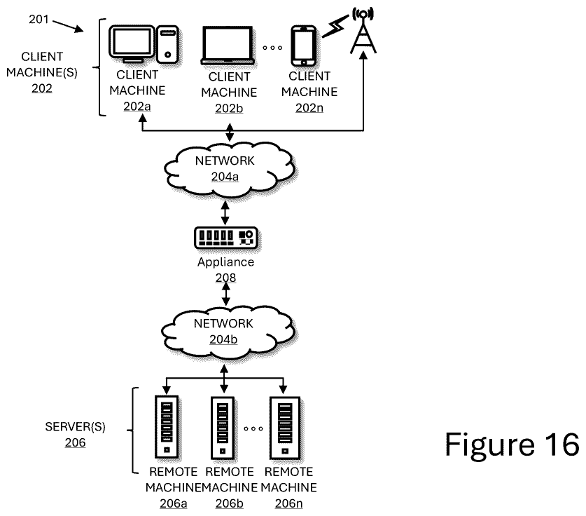

It is understood that aspects of the described infrastructure can be implemented in any manner, e.g., as a stand-alone system, a distributed system, within a network environment, etc. Referring to FIG. 16, a non-limiting network environment 201 in which various aspects of the disclosure may be implemented includes one or more client machines 202A-202N, one or more remote machines 206A-206N, one or more networks 204, 204′, and one or more appliances 208 installed within the computing environment 201. The client machines 202A-202N communicate with the remote machines 206A-206N via the networks 204, 204′.

In some embodiments, the client machines 202A-202N communicate with the remote machines 206A-206N via an intermediary appliance 208. The illustrated appliance 208 is positioned between the networks 204, 204′ and may also be referred to as a network interface or gateway. In some embodiments, the appliance 208 may operate as an application delivery controller (ADC) to provide clients with access to business applications and other data deployed in a datacenter, the cloud, or delivered as Software as a Service (SaaS) across a range of client devices, and/or provide other functionality such as load balancing, etc. In some embodiments, multiple appliances 208 may be used, and the appliance(s) 208 may be deployed as part of the network 204 and/or 204′.

The client machines 202A-202N may be generally referred to as client machines 202, local machines 202, clients 202, client nodes 202, client computers 202, client devices 202, computing devices 202, endpoints 202, or endpoint nodes 202. The remote machines 206A-206N may be generally referred to as servers 206 or a server farm 206. In some embodiments, a client device 202 may have the capacity to function as both a client node seeking access to resources provided by a server 206 and as a server 206 providing access to hosted resources for other client devices 202A-202N. The networks 204, 204′ may be generally referred to as a network 204. The networks 204 may be configured in any combination of wired and wireless networks.

A server 206 may be any server type such as, for example: a file server; an application server; a web server; a proxy server; an appliance; a network appliance; a gateway; an application gateway; a gateway server; a virtualization server; a deployment server; a Secure Sockets Layer Virtual Private Network (SSL VPN) server; a firewall; a web server; a server executing an active directory; a cloud server; or a server executing an application acceleration program that provides firewall functionality, application functionality, or load balancing functionality.

A server 206 may execute, operate or otherwise provide an application that may be any one of the following: software; a program; executable instructions; a virtual machine; a hypervisor; a web browser; a web-based client; a client-server application; a thin-client computing client; an ActiveX control; a Java applet; software related to voice over internet protocol (VoIP) communications like a soft IP telephone; an application for streaming video and/or audio; an application for facilitating real-time-data communications; a HTTP client; a FTP client; an Oscar client; a Telnet client; or any other set of executable instructions.

In some embodiments, a server 206 may execute a remote presentation services program or other program that uses a thin-client or a remote-display protocol to capture display output generated by an application executing on a server 206 and transmit the application display output to a client device 202.

In yet other embodiments, a server 206 may execute a virtual machine providing, to a user of a client device 202, access to a computing environment. The client device 202 may be a virtual machine. The virtual machine may be managed by, for example, a hypervisor, a virtual machine manager (VMM), or any other hardware virtualization technique within the server 206.

In some embodiments, the network 204 may be: a local-area network (LAN); a metropolitan area network (MAN); a wide area network (WAN); a primary public network 204; and a primary private network 204. Additional embodiments may include a network 204 of mobile telephone networks that use various protocols to communicate among mobile devices. For short range communications within a wireless local-area network (WLAN), the protocols may include 802.11, Bluetooth, and Near Field Communication (NFC).

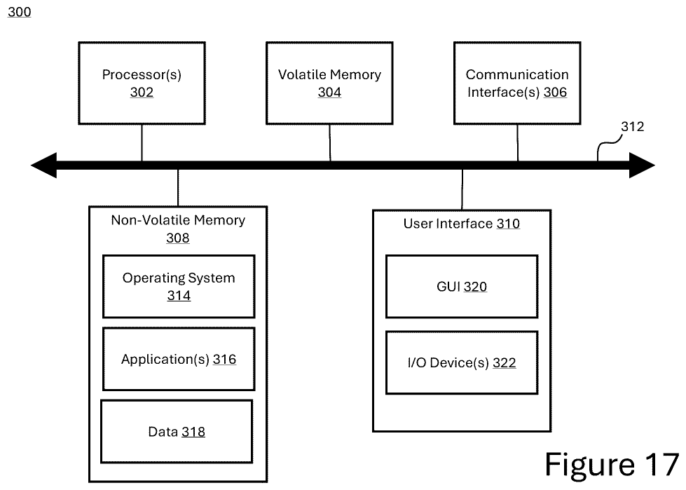

Elements of the described solution may be embodied in a computing system, such as that shown in FIG. 17 in which a computing device 300 may include one or more processors 302, volatile memory 304 (e.g., RAM), non-volatile memory 308 (e.g., one or more hard disk drives (HDDs) or other magnetic or optical storage media, one or more solid state drives (SSDs) such as a flash drive or other solid state storage media, one or more hybrid magnetic and solid state drives, and/or one or more virtual storage volumes, such as a cloud storage, or a combination of such physical storage volumes and virtual storage volumes or arrays thereof), user interface (UI) 310, one or more communications interfaces 306, and communication bus 312. User interface 310 may include graphical user interface (GUI) 320 (e.g., a touchscreen, a display, etc.) and one or more input/output (I/O) devices 322 (e.g., a mouse, a keyboard, etc.). Non-volatile memory 308 stores operating system 314, one or more applications 316, and data 318 such that, for example, computer instructions of operating system 314 and/or applications 316 are executed by processor(s) 302 out of volatile memory 304. Data may be entered using an input device of GUI 320 or received from I/O device(s) 322. Various elements of computer 300 may communicate via communication bus 312. Computer 300 as shown in FIG. 17 is shown merely as an example, as clients, servers and/or appliances and may be implemented by any computing or processing environment and with any type of machine or set of machines that may have suitable hardware and/or software capable of operating as described herein.

Processor(s) 302 may be implemented by one or more programmable processors executing one or more computer programs to perform the functions of the system. As used herein, the term “processor” describes an electronic circuit that performs a function, an operation, or a sequence of operations. The function, operation, or sequence of operations may be hard coded into the electronic circuit or soft coded by way of instructions held in a memory device. A “processor” may perform the function, operation, or sequence of operations using digital values or using analog signals. In some embodiments, the “processor” can be embodied in one or more application specific integrated circuits (ASICs), microprocessors, digital signal processors, microcontrollers, field programmable gate arrays (FPGAs), programmable logic arrays (PLAs), multi-core processors, or general-purpose computers with associated memory. The “processor” may be analog, digital or mixed-signal. In some embodiments, the “processor” may be one or more physical processors or one or more “virtual” (e.g., remotely located or “cloud”) processors.

Communications interfaces 306 may include one or more interfaces to enable computer 300 to access a computer network such as a LAN, a WAN, or the Internet through a variety of wired and/or wireless or cellular connections.

In described embodiments, a first computing device 300 may execute an application on behalf of a user of a client computing device (e.g., a client), may execute a virtual machine, which provides an execution session within which applications execute on behalf of a user or a client computing device (e.g., a client), such as a hosted desktop session, may execute a terminal services session to provide a hosted desktop environment, or may provide access to a computing environment including one or more of: one or more applications, one or more desktop applications, and one or more desktop sessions in which one or more applications may execute.

As will be appreciated by one of skill in the art upon reading the following disclosure, various aspects described herein may be embodied as a system, a device, a method or a computer program product (e.g., a non-transitory computer-readable medium having computer executable instruction for performing the noted operations or steps).

Accordingly, those aspects may take the form of an entirely hardware embodiment, an entirely software embodiment, or an embodiment combining software and hardware aspects. Furthermore, such aspects may take the form of a computer program product stored by one or more computer-readable storage media having computer-readable program code, or instructions, embodied in or on the storage media. Any suitable computer readable storage media may be utilized, including hard disks, CD-ROMs, optical storage devices, magnetic storage devices, and/or any combination thereof.

The foregoing drawings show some of the processing associated according to several embodiments of this disclosure. In this regard, each drawing or block within a flow diagram of the drawings represents a process associated with embodiments of the method described. It should also be noted that in some alternative implementations, the acts noted in the drawings or blocks may occur out of the order noted in the figure or, for example, may in fact be executed substantially concurrently or in the reverse order, depending upon the act involved. Also, one of ordinary skill in the art will recognize that additional blocks that describe the processing may be added.

What is claimed is:

1. A dental collaboration system, comprising:
a virtual practice private network (VPPN) service configured to establish a unique VPPN for each of a plurality of dental practices, wherein each VPPN grants access rights to a unique set of users;
an adaptable integration framework (AIF) configured to provide read and write capabilities to disparate practice management systems utilized by the plurality of dental practices;
a universal clinical toolset service configured to allow client applications to interact with data from the disparate practice management systems via a universal dental interface (UDI), wherein the UDI includes a system agnostic charting interface, a system agnostic notes interface, a system agnostic periodontics interface, and a system agnostic imaging interface; and
a collaboration service configured to allow the unique set of users of an associated VPPN to capture and share information in a collaboration interface within the UDI.

2. The system of claim 1, wherein the access rights granted to the unique set of users for an associated VPPN determines which of the disparate practice management systems are accessible to the unique set of users.

3. The system of claim 1, wherein the AIF utilizes one of application programming interfaces (APIs) or robotic process automation (RPA) to read and write data to disparate practice management systems.

4. The system of claim 1, wherein the system agnostic charting interface overlays patient specific image data onto a generic graphical image of a set of teeth based on patient records obtained via the AIF.

5. The system of claim 1, wherein the collaboration interface provides an active capture facility for recording and sharing screen displays, annotations and voice recordings within the unique set of users for the associated VPPN.

6. The system of claim 5, wherein the active capture facility can record and share screen displays presented in the UDI.

7. The system of claim 5, wherein the collaboration interface includes a messaging facility for sharing messages with other users in the unique set of users for the associated VPPN.

8. The system of claim 1, wherein at least one user of the unique set of users for the associated VPPN is an external specialist or lab that can only access patient records from an associated practice management system via the UDI.

9. The system of claim 1, further comprising a data normalizer that converts patient records from the disparate practice management systems to a common format utilized by the UDI.

10. The system of claim 9, wherein the data normalizer includes a dental code normalizer that maps vendor code extensions and practice code extensions to a normalized format.

11. A cloud computing infrastructure, comprising:
a memory; and
a processor coupled to the memory and configured to provide dental collaboration according to a process that includes:
configuring a unique VPPN for each of a plurality of dental practices, wherein each VPPN grants access rights to a unique set of users;
providing an adaptable integration framework (AIF) configured to provide read and write capabilities to disparate practice management systems utilized by the plurality of dental practices;
communicating dental practice data with client applications via a universal dental interface (UDI), wherein the dental practice data include patient records obtained from the disparate practice management systems, and wherein the UDI includes a system agnostic charting interface, a system agnostic notes interface, a system agnostic periodontics interface, and a system agnostic imaging interface; and
sharing information among the unique set of users of an associated VPPN via a collaboration interface within the UDI.

12. The infrastructure claim 11, wherein the access rights granted to the unique set of users for an associated VPPN determines which of the disparate practice management systems are accessible to the unique set of users.

13. The infrastructure claim 11, wherein the AIF utilizes one of application programming interfaces (APIs) or robotic process automation (RPA) to read and write data to disparate practice management systems.

14. The infrastructure claim 11, wherein the system agnostic charting interface overlays patient specific image data onto a generic graphical image of a set of teeth based on patient records obtained via the AIF.

15. The infrastructure claim 11, wherein the collaboration interface provides an active capture facility for recording and sharing screen displays, annotations and voice recordings among the unique set of users for the associated VPPN.

16. The infrastructure claim 15, wherein the active capture facility can record and share screen displays presented in the UDI.

17. The infrastructure claim 15, wherein the collaboration interface includes a messaging facility for sharing messages with other users in the unique set of users within the associated VPPN.

18. The infrastructure claim 11, wherein at least one user of the unique set of users for the associated VPPN is an external specialist or lab that can only access patient records from an associated practice management system via the UDI.

19. The infrastructure claim 11, further comprising converting patient records from the disparate practice management systems to a common format utilized by the UDI.

20. A method of providing dental collaboration, comprising:
configuring a unique VPPN for each of a plurality of dental practices, wherein each VPPN grants access rights to a unique set of users;
providing an adaptable integration framework (AIF) configured to provide read and write capabilities to disparate practice management systems utilized by the plurality of dental practices;
communicating dental practice data with client applications via a universal dental interface (UDI), wherein the dental practice data include patient records obtained from the disparate practice management systems, and wherein the UDI includes a system agnostic charting interface, a system agnostic notes interface, a system agnostic periodontics interface, and a system agnostic imaging interface; and
sharing information among the unique set of users of an associated VPPN via a collaboration interface within the UDI.
